(19) United States
(12) Patent Application Publication
(10) Pub. No.: US 2020/0384097 A1
(43) Pub. Date: Dec. 10, 2020

(54) COMPOSITION

(71) Applicants: University of Exeter, Devon (GB); Universiteit Gent, Ghent (BE)

(72) Inventors: Richard William Titball, Devon (GB); Sergio Paulo Fernandes Da Costa, Frankfurt am Main (DE); Filip Van Immerseel, Eke (BE); Richard Ducatelle, Wortegem-Petegem (BE); Dorien Mot, Waarbeke (BE)

(21) Appl. No.: 16/997,484

(22) Filed: Aug. 19, 2020

Related U.S. Application Data
(62) Division of application No. 15/106,199, filed on Jun. 17, 2016, now abandoned.

(30) Foreign Application Priority Data
Dec. 18, 2013 (GB) 1322463.9

(51) Int. Cl. A61K 39/08; C07K 14/33

(52) U.S. Cl. CPC A61K 39/08; A61K 2039/552; C07K 14/33

(57) ABSTRACT

The invention provides a composition comprising a reduced toxicity NetB epitope polypeptide and a reduced toxicity Clostridium perfringens alpha-toxin epitope polypeptide. The composition is useful as a vaccine providing complete protection against infection by C. perfringens.

### CROSS REFERENCE TO RELATED APPLICATIONS

This application is a divisional of U.S. application Ser. No. 15/106,199, filed Jun. 17, 2016, which is a national stage application under 35 U.S.C. § 371 of International Application No. PCT/GB2014/053748, filed Dec. 18, 2014, entitled “COMPOSITION,” which claims priority to Patent Application No. GB1322463.9, filed Dec. 18, 2013.

### INCORPORATION BY REFERENCE OF SEQUENCE LISTING

This application contains a Sequence Listing that has been submitted in ASCII format via EFS-Web and is hereby incorporated by reference in its entirety. The ASCII copy, created on Dec. 18, 2014, is named P2299PC00_Sequence_Listing.txt, and is about 20,000 bytes in size.

### FIELD OF THE INVENTION

The invention relates to a composition comprising two or more polypeptides which, when administered together, provide complete protection to a subject against infection by Clostridium perfringens.

### BACKGROUND

Clostridium perfringens (C. perfringens) is a ubiquitous bacterium that is able to colonise a variety of different biotopes and it is not unusual to find C. perfringens as a commensal in the normal gut microbiota in domesticated animals. However, under particular circumstances the bacterium is responsible for severe diseases. These diseases are largely a consequence of the actions of toxins on the host (Songer, Clin Microbiol Rev 1996; 9(2):216-34). In addition to the four toxins used for typing C. perfringens strains (alpha-, beta-, epsilon-, and iota-toxin), the bacterium is able to produce a number of other toxins, including enterotoxin and perfringolysin O (McDonel, Pharmacol Ther 1980; 10(3):617-55 and Petit et al., Trends Microbiol 1999; 7(3):104-10).

C. perfringens has been shown to cause avian necrotic enteritis (NE), a severe gastro-intestinal disease of farmed poultry (Cooper et al., Vet Microbiol 2009; 142(3-4):323-8, Parish, J Comp Pathol 1961; 71:377-93 and Keyburn et al., PLoS Pathog 2008 Feb. 8; 4(2):e26). Until recently, NE has been controlled by the addition of antimicrobial growth promoters to feedstuffs. However, in many countries national and supranational regulations now limit the addition of antimicrobials to animal feeds. Consequently, in these countries, NE is emerging as a disease which is of significant economic consequence to the poultry industry (Van Der Sluis, World Poultry 2000; 16:42-3 and Castanon, Poult Sci 2007; 86(11):2466-71). The disease can occur in at least two forms. The acute form of NE typically results in mortality during the last weeks of rear of broilers (week 5-6). However, many cases of NE are associated with relatively mild clinical signs (Kaldhusdal et al., Poul Sci 1992; 71:1145-53, Brennan et al., Poult Sci 2001 October; 80(10):1451-4 and Brennan et al., Avian Dis 2001 January-March; 45(1):210-4). This subclinical form of NE results in decreased digestion and absorption of feedstuffs and consequently reduced weight gain (Elwinger et al., Acta Vet Scand 1992; 33(4):369-78 and Kaldhusdal et al., Avian Dis 2001 January-March; 45(1):149-56). At least in Europe it is now believed that the subclinical NE is the most frequent form of the disease and causes the greatest economic losses to the poultry production industry (Dahiya et al., Animal Feed Sci Technol 2006; 129:60-88).

Although it is clear that C. perfringens is the etiologic agent of NE, a wide range of host and pathogen factors can influence the severity of the disease. These factors include the nature of the feedstuff, co-infection with various Eimeria species and the molecular makeup of C. perfringens in the gut (Shojadoost et al., Vet Res 2012 Oct. 26; 43(1):74). Often these factors interact with each other, and this has made the development of reliable infection models difficult (Shojadoost et al., Vet Res 2012 Oct. 26; 43(1):74). The molecular basis of virulence of C. perfringens associated with NE is still being investigated. However, almost all C. perfringens isolates from cases of NE possess the netB gene (Cooper et al., Vet Microbiol 2009; 142(3-4):323-8, Chalmers et al., J Clin Microbiol 2008; 46(12):3957-64 and Martin et al., Vet Microbiol 2009; 136(1-2):202-5) which encodes necrotic enteritis toxin B (NetB), a β-pore-forming toxin (Keyburn et al., PLoS Pathog 2008 Feb. 8; 4(2):e26 and Savva et al., J Biol Chem 2013 Feb. 1; 288(5):3512-22). Pore formation by NetB can lead to cell lysis by disruption of membrane integrity and a netB mutant of C. perfringens is reported to be incapable of causing NE (Keyburn et al., PLoS Pathog 2008 Feb. 8; 4(2):e26). There is also accumulating evidence that other virulence factors, such as the TpeL toxin, play a role in disease (Coursodon et al., Anaerobe 2012 February; 18(1):117-21). The production of these virulence factors might explain the reported ability of some netB-negative strains of C. perfringens to cause NE (Cooper et al., Vet Microbiol 2009; 142(3-4):323-8 and Keyburn et al., PLoS Pathog 2008 Feb. 8; 4(2):e26).

Immunisation with either crude toxoids (Saleh et al., Anaerobe 2011; 17(6):426-30) or culture supernatants (Lanckriet et al., Vaccine 2010; 28(36):5920-3) can provide significant but incomplete protection against experimental NE. Although these vaccines are simple to prepare they suffer from the limitation that it is difficult to configure them for non-invasive dosing, for example by oral delivery. Other workers have explored the possibility of a sub-unit which is able to protect against NE, with a view towards both improving vaccine efficacy and opening the possibility of oral delivery. To date, a range of proteins derived from C. perfringens have been evaluated as sub-unit vaccines including alpha-toxin, glyceraldehyde-3-phosphate dehydrogenase, pyruvate-ferredoxin oxidoreductase, fructose 1,6-biphosphate-aldolase, or a hypothetical protein (Kulkarni et al., Clin Vaccine Immunol 2007; 14(9):1070-7). Immunisation with any of these sub-units provided partial protection against experimental NE. Partial protection against NE has also been reported after immunisation with C. perfringens large cytotoxin TpeL, endo-beta-N-acetylglucosaminidase or phosphoglyceromutase (Jiang et al., Avian Dis 2009; 53(3):409-15). A more recent study in which alpha-toxin, NetB, pyruvate-ferredoxin oxidoreductase and elongation factor-Tu were compared as protective antigens concluded that NetB and pyruvate-ferredoxin oxidoreductase given with ISA71 adjuvant provided enhanced protective immunity (Jang et al., Vaccine 2012; 30(36):5401-6). However, it is unlikely that a licensed vaccine for widespread use could contain active toxins. Therefore, there is a need to identify non-toxic variants of these toxins.

It has previously been shown that a NetB mutant (W262A) was able to induce partial protection against experimental NE in poultry (Fernandes da Costa et al., Vaccine 2013 Aug. 20; 31(37):4003-8 and WO2013/061056). The C-terminal region of the alpha-toxin has also been investigated for this purpose (WO93/23543).

### SUMMARY OF THE INVENTION

According to a first aspect of the invention, there is provided a composition comprising a reduced toxicity NetB epitope polypeptide and a reduced toxicity Clostridium perfringens alpha-toxin epitope polypeptide.

Surprisingly, the present inventors have found that administration of both a reduced toxicity NetB epitope polypeptide and a reduced toxicity C. perfringens alpha-toxin epitope polypeptide to chickens confers 100% protection against infection by C. perfringens. This is unexpected in view of the outcome of administration of C. perfringens culture supernatants to animals, in which supernatants comprising both wild-type NetB and alpha-toxin varied in their ability to confer protection (Lanckriet et al., Vaccine 2010; 28(36):5920-3).

The term “NetB epitope polypeptide” as used throughout this specification means a polypeptide which comprises one or more (or all) epitopes of mature wild-type NetB, as represented by the amino acid SEQ ID NO:1. The term “Clostridium perfringens alpha-toxin epitope polypeptide” as used throughout this specification means a polypeptide which comprises one or more (or all) epitopes of the C. perfringens alpha-toxin, as represented by the amino acid SEQ ID NO:18. For example, it may comprise one or more (or all) epitopes of the C-terminal portion of C. perfringens alpha-toxin, as represented by the amino acid SEQ ID NO:3) (and as disclosed in WO93/23543 as SEQ ID NO:4). The term “epitope” refers to the amino acids (typically a group of around 5 or more amino acids) within a polypeptide sequence which are essential in the generation of an immune response. These amino acids can be consecutive in the sequence but, more typically, are non-consecutive, grouping together when the tertiary structure of the native protein is formed. For example, a NetB epitope polypeptide may be one which is capable of binding to an antibody which binds to the mature wild-type NetB having sequence SEQ ID NO:1, while a C. perfringens alpha-toxin epitope polypeptide may be one which is capable of binding to an antibody which binds to C. perfringens alpha-toxin, i.e., SEQ ID NO:18 (for example, an antibody which binds to SEQ ID NO:3).

An epitope polypeptide may be, as mentioned above, any which comprises at least one epitope of the polypeptide referred to and is capable of binding an antibody which will bind to the polypeptide referred to, that is, NetB (SEQ ID NO:1) or C. perfringens alpha-toxin (SEQ ID NO:18). Therefore, the polypeptide may be as little as about 20 amino acids in length provided that it still binds to such an antibody, for example, it may be at least about 30, 40, 50, 60, 70, 80, 90 or 100 amino acids in length. Both the NetB wild-type polypeptide and the alpha-toxin wild-type polypeptide are initially expressed in the cell with a 30- or 28-amino acid N-terminal signal sequence, respectively; the epitope polypeptide may comprise such a signal sequence.

A NetB or alpha-toxin epitope polypeptide being of “reduced toxicity”, as referred to throughout this specification, indicates a reduced toxicity as compared to the mature wild-type NetB having sequence SEQ ID NO:1 or mature wild-type alpha-toxin having SEQ ID NO:18, respectively. The level of toxicity may be determined as described herein and in co-pending application PCT/GB2012/052639 (WO2013/061056), for example by use of a chicken hepatocellular carcinoma (LMH) cell-based lactate dehydrogenase (LDH) assay. The level of toxicity should be below that which causes effects in poultry. This can be readily assessed using methods known in the art. In one embodiment, the reduced toxicity is not a function of chemical treatment. In one embodiment, the reduced toxicity is caused by alteration of the polypeptide by substitution, deletion or addition of one or more (i.e. 1, 2, 3, 4, 5, 6, 7, 8, 9, 10 or more) amino acids compared to the wild type sequence.

In this application, the NetB epitope polypeptide may comprise a sequence of at least 10 contiguous amino acids from SEQ ID NO:1, the sequence containing at least one amino acid substitution compared to the equivalent sequence of SEQ ID NO:1. For example, the amino acid substitution may be selected from:
a) W262X and W257X, wherein X is any amino acid other than W;
b) Y78X, Y182X, Y187X, Y191X and Y202X, wherein X is any amino acid other than Y;
c) P138X, wherein X is any amino acid other than P;
d) H188X, wherein X is any amino acid other than H; and
e) R200X, wherein X is any amino acid other than R.

The amino acid numbers given above for the substitution positions are with reference to the positions found in SEQ ID NO:1. Where a sequence shorter than SEQ ID NO:1 and comprising at least 10 contiguous amino acids from SEQ ID NO:1 is utilised, the position of the substitution is determined by aligning the rest of the shorter sequence with the sequence of SEQ ID NO:1 so that complete sequence identity is achieved between the aligned sequences, with the exception of the substitution point. Such alignment can readily be achieved using a global sequence alignment program such as the Needleman-Wunsch Global Sequence Alignment Tool, discussed in more detail elsewhere herein.

The amino acid substitutions may be particularly selected from W262X, Y191X, R200X or W257X. In any of the above substitutions, X may be A (alanine). In one embodiment, the substitution is W262A.

In an embodiment, the NetB epitope polypeptide comprises at least one of the sequences:
a) ETTQXRGTNK (SEQ ID NO:10) (for example, the polypeptide may comprise SEQ ID NO:11), wherein X is any amino acid other than W;
b) YHAIXGNQLF (SEQ ID NO:4) (for example, the polypeptide may comprise SEQ ID NO:5), wherein X is any amino acid other than Y;
c) FMKSXLYNNG (SEQ ID NO:6) (for example, the polypeptide may comprise SEQ ID NO:7), wherein X is any amino acid other than R; or
d) YILNXETTQW (SEQ ID NO:8) (for example, the polypeptide may comprise SEQ ID NO:9), wherein X is any amino acid other than W.

Each of these sequences comprises X at a substitution position mentioned above, with SEQ ID NOs:4 and 5 comprising position Y191, SEQ ID NOs:6 and 7 comprising position R200, SEQ ID NOs:8 and 9 comprising W257 and SEQ ID NOs:10 and 11 comprising W262. In an embodiment, X in any of the sequences is A (alanine).

In an embodiment, in the composition according to the invention, the NetB epitope polypeptide comprises the amino acid sequence SEQ ID NO:2, or a functional fragment or variant thereof having at least 75% global sequence identity to SEQ ID NO:2. SEQ ID NO:2 is the amino acid sequence of NetB comprising substitution W262A. Preparation of this polypeptide is described in co-pending application PCT/GB2012/052639 (WO2013/061056). A functional fragment or variant of SEQ ID NO:2 is a fragment or variant which is capable of binding to an antibody which will bind to wild-type NetB (SEQ ID NO:1) and which has similar toxicity to SEQ ID NO:2, for example assessed by use of a LMH cell-based LDH assay as outlined above. “Similar toxicity” may be, for example, toxicity within about 5%, 10%, 15% or about 20% of the toxicity of SEQ ID NO:2 itself. The functional fragment or variant may have global sequence identity, for example assessed using the Needleman-Wunsch Global Sequence Alignment Tool described elsewhere herein, of at least about 75%, 80%, 85%, 90%, 91%, 92%, 93%, 94%, 95%, 96%, 97%, 98% or at least about 99%. For example, the functional fragment may have up to 73 amino acids removed from the N- or C-terminal ends of the polypeptide, for example, up to about 10, 20, 30, 40, 50, 60 or about 70 amino acids removed. This figure may be the total number of amino acids removed from the polypeptide, with some of the total number removed from the C-terminal end and some of the total number removed from the N-terminal end. The functional fragment or variant may comprise the amino acid sequence ETTQARGTNK (SEQ ID NO:17), which is a ten amino acid sequence comprising amino acid A at the position equivalent to position 262 of the full length polypeptide (SEQ ID NO:2) when SEQ ID NOs:2 and 17 are aligned. In one embodiment, a variant of a NetB epitope polypeptide has one or more (i.e. 1, 2, 3, 4, 5, 6, 7, 8, 9, 10 or more) amino acid substitutions, deletions or additions when compared to SEQ ID NO:2.

In this application, the alpha-toxin epitope polypeptide may comprise a sequence of at least 5 contiguous amino acids from SEQ ID NO:18. For example, it may comprise a sequence of at least 5 contiguous amino acids from SEQ ID NO:3, the sequence containing sequences DDY and/or PGN. SEQ ID NO:3 is the amino acid sequence of the C-terminal region (residues 247-370) of the alpha-toxin polypeptide disclosed as SEQ ID NO:2 in WO93/23542. The residues 273-275 (DDY) and 295-297 (PGN) of the full-length polypeptide have previously been identified as the positions of some of the C-terminal epitopes (WO93/23543; Logan et al. (1991) Infect. Immun. vol. 59, p 4338-4342).

For example, the alpha-toxin epitope polypeptide may comprise the amino acid sequence SEQ ID NOs:12 and/or 13 and/or 14 and/or 15. SEQ ID NOs:12 and 13 are found in SEQ ID NO:3 and comprise the DDY position; SEQ ID NOs: 14 and 15 are found in SEQ ID NO:3 and comprise the PGN position. The alpha-toxin epitope polypeptide may comprise the amino acid sequence SEQ ID NO:16; this is found in SEQ ID NO:3 and comprises the DDY position and the PGN position and all of SEQ ID NOs: 12-15.

In an embodiment, the alpha-toxin epitope polypeptide comprises the amino acid sequence SEQ ID NO:18, or a functional fragment or variant thereof having at least 34% global sequence identity to SEQ ID NO:18. A functional fragment or variant of SEQ ID NO:18 is a fragment or variant which is capable of binding to an antibody which will bind to SEQ ID NO:18. The functional fragment or variant may have global sequence identity, for example assessed using the Needleman-Wunsch Global Sequence Alignment Tool described elsewhere herein, of at least about 40%, 45%, 50%, 55%, 60%, 65%, 70%, 75%, 80%, 85%, 90%, 91%, 92%, 93%, 94%, 95%, 96%, 97%, 98% or about 99%. For example, the functional fragment may have up to 246 amino acids removed from one of the ends of the polypeptide.

For example, amino acid residues 1-246 may be removed from the N-terminal end of SEQ ID NO:18, with residues 247-370 remaining as SEQ ID NO:3 (which, at a global level, is 34% identical to SEQ ID NO:18), which may form the alpha-toxin epitope polypeptide referred to herein. The alpha-toxin epitope polypeptide may be a functional fragment or variant of SEQ ID NO:3, having at least 75% global sequence identity thereto. A functional fragment or variant of SEQ ID NO:3 is a fragment or variant which is capable of binding to an antibody which will bind to SEQ ID NO:3. The functional fragment or variant may have global sequence identity, for example assessed using the Needleman-Wunsch Global Sequence Alignment Tool described elsewhere herein, of at least about 75%, 80%, 85%, 90%, 91%, 92%, 93%, 94%, 95%, 96%, 97%, 98% or about 99%. For example, the functional fragment may have up to 31 amino acids removed from the N- or C-terminal ends of the polypeptide, for example, up to about 5, 10, 15, 20, 25 or about 30 amino acids removed. This figure may be the total number of amino acids which are removed from the polypeptide, with some of the total number removed from the C-terminal end and some of the total number removed from the N-terminal end. In one embodiment, a variant of an alpha-toxin epitope polypeptide has one or more (i.e. 1, 2, 3, 4, 5, 6, 7, 8, 9, 10 or more) amino acid substitutions, deletions or additions when compared to SEQ ID NO:18.

As mentioned, the present invention encompasses a composition comprising variants of the epitope polypeptides and methods utilising these variant polypeptides. As used herein, a “variant” means a polypeptide in which the amino acid sequence differs from the base sequence from which it is derived in that one or more amino acids (i.e. 1, 2, 3, 4, 5, 6, 7, 8, 9, 10 or more) within the sequence are substituted for other amino acids, or are deleted or added. The variant is a functional variant, in that the functional characteristics of the polypeptide from which the variant is derived are maintained. For example, the variant polypeptide may have a similar ability to bind an antibody capable of binding to a non-variant polypeptide (such as SEQ ID NO:1, 18 or 3). In particular, any amino acid substitutions, additions or deletions must not alter or significantly alter the tertiary structure of one or more epitopes contained within the polypeptide from which the variant is derived, so that the variant polypeptide retains the ability to bind to an antibody which binds to SEQ ID NO:1, 18 or 3, as appropriate. The skilled person is readily able to determine appropriate functional variants and to determine the tertiary structure of an epitope and any alterations thereof, without the application of inventive skill.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | Ala, Val, Leu, Ile, Pro, Met, Phe, Trp |
| Uncharged polar: | Gly, Ser, Thr, Cys, Tyr, Asn, Gln |
| Acidic: | Asp, Glu |
| Basic: | Lys, Arg, His. |

As is well known to the skilled person, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

As mentioned above, non-conservative substitutions are possible provided that these do not disrupt the tertiary structure of an epitope within the polypeptide, for example, which do not interrupt the immunogenicity (for example, the antigenicity) of the polypeptide.

Broadly speaking, fewer non-conservative substitutions will be possible without altering the biological activity of the polypeptide. As mentioned above, variants may suitably be at least about 75% identical to the base sequence.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

As mentioned previously, sequence identity preferably is determined using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings. When comparing the level of sequence identity to SEQ ID NO:1, for example, this typically should be done relative to the whole length of SEQ ID NO:1, to avoid short regions of high identity overlap resulting in a high overall assessment of identity (i.e., a global alignment method is used).

In the composition according to the invention, at least one polypeptide is a recombinant polypeptide. That is, the polypeptide is not naturally occurring, but has been prepared using established laboratory techniques, as outlined, for example, in Green & Sambrook (Molecular Cloning, a laboratory manual [fourth edition] Green & Sambrook, Cold Spring Harbor Laboratory, 2012). Such techniques are well known to the skilled person.

Polypeptides used with the invention are preferably provided in purified or substantially purified form i.e. substantially free from other polypeptides (e.g. free from naturally-occurring polypeptides), particularly from other Clostridium or host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the polypeptides in the compositions are separated from the whole organism with which the molecule is expressed.

The alpha-toxin epitope polypeptide may be expressed as a fusion protein with Glutathione-S-Transferase (GST) or with a Histidine tag (His-tag). The NetB epitope polypeptide may be expressed with a Histidine tag (His-tag) or a fusion protein with Glutathione-S-Transferase (GST). Other expression tags may readily be utilised by the skilled person without application of inventive skill.

The composition according to the invention may comprise a fusion protein comprising a reduced toxicity NetB epitope polypeptide as described above and a reduced toxicity C. perfringens alpha-toxin epitope polypeptide as described above. Optionally, such a fusion protein may comprise a linker sequence between the two epitope polypeptides. Suitable linker amino acid sequences will be apparent to those skilled in the art, but include poly-glycine linkers (i.e. comprising Glyₙ where n=2, 3, 4, 5, 6, 7, 8, 9, 10 or more), and GSGGGG (SEQ ID NO: 21), with the Gly-Ser dipeptide being formed from a BamHI restriction site, thus aiding cloning and manipulation, and the (Gly)₄ tetrapeptide being a typical poly-glycine linker.

The composition may be a simple mixture of polypeptides in solution, for example in a buffer solution, or may form part of a composition comprising other components, such as in a vaccine composition as described below.

According to a second aspect of the invention, there is provided a polynucleotide encoding a reduced toxicity NetB epitope polypeptide and a reduced toxicity C. perfringens alpha-toxin epitope polypeptide. Therefore, a single polynucleotide sequence comprises a polynucleotide encoding for each polypeptide; these polynucleotides may be immediately adjacent one another or may be separated by a further polynucleotide sequence not encoding either polypeptide. The polynucleotide may be contained within a vector, which forms a third aspect of the invention. The vector may comprise additional expression control sequences such as at least one promoter sequence operably linked to the sequence(s) encoding for the polypeptides, terminator region and one or more inducer regions. The polynucleotide encoding the NetB epitope polypeptide may be under the control of a separate promoter region to the polynucleotide encoding the alpha-toxin epitope polypeptide. The design of a suitable vector is within the routine abilities of the skilled person, for example, with reference to Green & Sambrook (Molecular Cloning, a laboratory manual [fourth edition] Green & Sambrook, Cold Spring Harbor Laboratory, 2012).

A fourth aspect of the invention provides a cell comprising a composition according to the first aspect, a polynucleotide according to the second aspect and/or a vector according to the third aspect of the invention. For example, a suitable cell may be a Salmonella cell, such as a Salmonella enterica cell, in some embodiments from the serovar typhimurium. The Salmonella may be an attenuated strain. Strains χ8914 and χ9241 may optionally be employed. Such cells are particularly useful to act as vectors when the polypeptide, polynucleotide and vector of the invention is to be used to provide a vaccine for chickens, to reduce the probability that they will be susceptible to infection by C. perfringens. For example, such a system is described in Kulkarni et al., Vaccine 2008 Aug. 5; 26(33):4194-203.

Other suitable cells may include attenuated mutants of Bacillus species or of C. perfringens. Inclusion of the polypeptides in a bacterium that is normally a member of the subject's gut flora is also contemplated. Lactococcus and Lactobacillus species are also contemplated.

A fifth aspect of the invention provides a vaccine composition comprising a composition according to the first aspect and/or a polynucleotide according to the second aspect and/or a vector according to the third aspect and/or a cell according to the fourth aspect of the invention. The vaccine composition may comprise a subunit vaccine, for example, in the form of a fusion protein and/or a recombinant viral vaccine.

The vaccine composition may further comprise excipients and/or diluents appropriate for the means by which the composition is to be administered to a subject in need of vaccination against infection by C. perfringens. Selection of appropriate components is within the routine capability of the skilled person without the application of inventive activity.

For example, the vaccine composition of the invention may conveniently be formulated using a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobials, anti-oxidants, chelating agents and inert gases. The pH and exact concentration of the various components the vaccine composition are adjusted according to routine skills.

Optionally, the vaccine formulation may include a carrier. Commonly used carrier molecules are bovine serum albumin (BSA), keyhole limpet hemocyanin (KLH), ovalbumin, mouse serum albumin, rabbit serum albumin and the like. Synthetic carriers may be used and are readily available. Means for conjugating peptides to carrier proteins are well known in the art and include glutaraldehyde, m-maleimidobenzoyl-N-hydroxysuccinimide ester, carbodiimide and bis-biazotized benzidine.

In certain situations, it may also be desirable to formulate the vaccine composition to comprise an adjuvant to enhance the immune response. Such adjuvants include all acceptable immunostimulatory compounds such as, for example, a cytokine, toxin, or synthetic composition. Commonly used adjuvants include the mineral-containing compositions aluminium hydroxide, aluminium phosphate, calcium phosphate; oil emulsions including squalene-water emulsions such as MF59, complete and incomplete Freund's adjuvants, and the Montanide ISA series of animal adjuvants; saponin formulations including Quil-A saponin, such as QS21 marketed as Stimulon™, as well as lipid formulations, such as ISCOMs; bacterial or microbial derivatives, such as non-toxic derivatives of enterobacterial lipopolysaccharide (LPS) including monophosphoryl lipid A (MPL) and 3-O-deacylated MPL (3dMPL); polymer adjuvants such as Montanide gel; as well as combinations thereof such as AS04 (comprising aluminium phosphate and MPL). In addition to adjuvants, it may be desirable to co-administer biologic response modifiers (BRM) with the peptide or variant or derivative to down regulate suppressor T cell activity.

Possible vehicles for administration of the vaccine formulation include liposomes. Liposomes are microscopic vesicles that consist of one or more lipid bilayers surrounding aqueous compartments. Liposomes are similar in composition to cellular membranes and, as a result, liposomes generally can be administered safely and are biodegradable. Techniques for preparation of liposomes and the formulation (e.g., encapsulation) of various molecules, including peptides and oligonucleotides, with liposomes are well known.

Depending on the method of preparation, liposomes may be unilamellar or multilamellar and can vary in size with diameters ranging from 0.02 μm to greater than 10 μm. Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents. In the present context, the polypeptides in the composition according to the first aspect of the invention can be localized on the surface of the liposome, to facilitate antigen presentation without disruption of the liposome or endocytosis. Irrespective of the mechanism or delivery, however, the result is the intracellular disposition of the associated polypeptides.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides.

Other suitable liposomes that are used in the compositions and methods of the invention include multilamellar vesicles (MLV), oligolamellar vesicles (OLV), unilamellar vesicles (UV), small unilamellar vesicles (SUV), medium-sized unilamellar vesicles (MIN), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). Techniques for preparing these liposomes are well known in the art.

Other forms of delivery particle, for example, microspheres and the like, also are contemplated for delivery of the epitope polypeptides.

Alternatively, nucleic acid-based vaccines may be produced that comprise nucleic acid, such as, for example, DNA or RNA, encoding the immunologically active epitope polypeptides and cloned into a suitable vector (e.g., vaccinia, canarypox, adenovirus, or other eukaryotic virus vector).

Compositions of the invention may be administered as a liquid, emulsion, dried powder and/or in a mist. Such compositions may be administered parenterally or enterally. If parenteral administration is desired, the compositions may be injected subcutaneously, intramuscularly, intraperitoneally or intradermally, or alternatively may be delivered by scarification or via a mucosal route such as intranasally, by aerosol or eye drop.

When intended for use with birds, the vaccine composition may be formulated to be suitable for in ovo administration, for example, via injection into the egg prior to hatching (e.g., into the amniotic fluid, the body of the embryo or the yolk sac). For example, injection into the amniotic fluid may be followed by ingestion by the embryo prior to hatching.

Alternatively, the composition may take the form of a cellular vaccine for delivery via the administration of autologous or allogeneic APCs or dendritic cells that have been treated in vitro so as to present the polypeptides on their surface. Salmonella cells may also be used, especially for administration to poultry, such as chickens. This involves the use of live attenuated Salmonella vaccines to deliver the antigens. This approach offers a number of advantages. First, live Salmonella vaccines can be given orally (the natural route of infection), enabling a non-invasive route of vaccine administration. Second, both mucosal and systemic immune responses can be elicited, which may be important for protection against infection. In addition, live attenuated Salmonella vaccines are able to simulate both humoral and cellular immune responses that may be important for protection against disease. Finally, since Salmonella is genetically tractable, recombinant Salmonella vaccines are relatively easy to develop and are also relatively cost effective to produce.

One of the most widely studied classes of attenuated Salmonella used as carriers of foreign antigens are auxotrophs. For example, genetically defined mutants of the aroA gene, encoding 5-enolpyruvylshikimate-3-phosphate synthase, have been constructed in both S. enterica var. Typhimurium and var. Typhi. These mutants are attenuated and immunogenic in mice. Examples of other auxotrophic mutants include Salmonella with deletions in the genes involved in the purine biosynthetic pathway. Another well-studied group of attenuated Salmonella are mutants that have defined deletions in genes involved in the regulation of Salmonella virulence. For example, mutations in genes encoding adenylate cyclase (cya) and camp receptor protein (crp) affect the expression of genes involved.

Other suitable cells may include attenuated mutants of Bacillus species or of C. perfringens. Inclusion of the polypeptides in a bacterium that is normally a member of the subject's gut flora is also contemplated. Lactococcus and Lactobacillus species are also contemplated.

In one embodiment, the vaccine composition may be included in an animal feed (i.e., a foodstuff suitable for consumption by an animal, particularly a chicken) comprising a composition and/or a polynucleotide and/or a vector and/or a cell and/or vaccine composition according to preceding aspects of the invention. This may, in non-limiting examples, be in the form of pellets, crumbs or a mash which may further comprise, again for example only, grain, grass and/or protein components. The composition may also be included in drinking liquids and/or administered via a spray into the atmosphere surrounding the animal which is, consequently, inhaled by the animal.

In a sixth aspect of the invention, there is provided a composition according to the first aspect, or a polynucleotide according the second aspect, or a vector according to the third aspect, or a cell according to the fourth aspect, or a vaccine composition according to the fifth aspect, for use in (i) therapy, (ii) eliciting an immune response against C. perfringens, and/or (iii) a method of vaccinating a subject against infection by Clostridium perfringens.

Likewise, a seventh aspect of the invention provides a method of (i) eliciting an immune response against C. perfringens and/or (ii) vaccinating a subject against infection by C. perfringens comprising administering to the subject a protective amount of a composition according to the first aspect, or a polynucleotide according to the second aspect, or a vector according to the third aspect, or a cell according to the fourth aspect, or a vaccine composition according to the fifth aspect of the invention.

A related eighth aspect of the invention providing a method of (i) eliciting an immune response against C. perfringens and/or (ii) vaccinating a subject against infection by C. perfringens may comprise administering to the subject a protective amount of a reduced toxicity NetB epitope polypeptide in combination with a protective amount of a reduced toxicity C. perfringens alpha-toxin epitope polypeptide. In the context of this aspect of the invention, the term “protective amount” of each polypeptide indicates an amount which is effective to vaccinate the subject against infection by C. perfringens when administered in combination with the other polypeptide. A related ninth aspect of the invention provides a reduced toxicity NetB epitope polypeptide in combination with a reduced toxicity C. perfringens alpha-toxin epitope polypeptide, for use in a method of vaccinating a subject against infection by C. perfringens. In such combinations, the component polypeptides may be administered simultaneously (i.e. by way of the same delivery device, or in the same feed) or sequentially (i.e. one after the other either using the same or different delivery devices/feeds). If administered sequentially, the polypeptides are preferably administered within about 24 hours or less (i.e. 18 hours, 12 hours, 6 hours, 3 hours, 1 hour, 45 minutes, 30 minutes, 15 minutes, 10 minutes, 5 minutes or less) of each other.

In both the seventh and eighth aspects, the term “protective amount” indicates an amount sufficient to induce an immune response in the subject, such that the probability of the subject becoming infected by C. perfringens if exposed to the bacterium is reduced or removed. For example, antibodies capable of binding to SEQ ID NO:1 and antibodies capable of binding to SEQ ID NO:18 and/or 3 may be detectable after the administration, where such antibodies were not detectable prior to the administration, or only detectable at lower concentrations than after administration.

In the sixth, seventh, eighth and ninth aspects, the subject may be poultry, such as chicken, turkey or duck. In one embodiment, the subject is of the genus Gallus, for example, of the species Gallus gallus (i.e., the domestic chicken). When the subject is a chicken, the preferred means for delivery of the polypeptide and/or a polynucleotide and/or a vector and/or a cell and/or a subunit vaccine of the other aspects of the invention may be a Salmonella-based system as described herein. The subject may also be a mammalian subject, for example, a human.

The term “vaccinating a subject”, as used herein, may indicate that the subject is completely protected from becoming infected by C. perfringens. That is, in any population vaccinated according to the described methods and/or using the described compositions or kits, less than 10%, preferably less than 5%, preferably 0% of individual subjects within that population will become infected by C. perfringens after exposure thereto. Infection may be assessed, for example, at least about 1, 2, 3, 4, 5, 6, 7, 10, 15, 21 or at least about 28 days after exposure of the subject to C. perfringens. One method of assessing infection is by using scoring lesions within the small intestine of each animal, as described by Keyburn et al., Infect Immun 2006 November; 74(11):6496-500. A non-invasive method of assessing infection is by monitoring the weight gain of each animal, wherein slower weight gain of a particular animal when compared to other similarly aged animals is indicative of infection. In one embodiment, the invention also provides a method of providing immunity to the progeny of a female mammal, comprising administering an effective amount of a composition of the invention to the female mammal prior to the birth of her progeny, whereby upon birth the progeny have acquired passive immunity to C. perfringens infection.

According to a tenth aspect of the invention, there is provided a kit comprising a composition and/or a polynucleotide and/or a vector and/or a cell and/or a vaccine composition according to any of the preceding aspects. In a related aspect, a kit may comprise a reduced toxicity NetB epitope polypeptide and a reduced toxicity C. perfringens alpha-toxin epitope polypeptide. For example, the kit may be a kit for use by a veterinarian or farmer to vaccinate a flock of chickens and may comprise a cellular vector (e.g., Salmonella or a Bacillus species etc., as described above) comprising a polypeptide according to the invention, for example for administration to chickens by inclusion in their feed. Instructions for use of the kit may be enclosed therewith. The kit may comprise a vial containing a composition and/or a polynucleotide and/or a vector and/or a cell and/or a vaccine composition according to any of the preceding aspects. The kit may also comprise a device for administration, for example, a syringe.

Throughout the description and claims of this specification, the words “comprise” and “contain” and variations of the words, for example “comprising” and “comprises”, mean “including but not limited to” and do not exclude other moieties, additives, components, integers or steps. The term “about” in relation to a numerical value x means, for example, x±10%.

Throughout the description and claims of this specification, the singular encompasses the plural unless the context otherwise requires. In particular, where the indefinite article is used, the specification is to be understood as contemplating plurality as well as singularity, unless the context requires otherwise.

Other features of the present invention will become apparent from the following examples. Generally speaking the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE FIGURES

The invention will now be described, by way of example only, with reference to FIGS. 1 and 2 in which:

FIG. 1 is a schematic outline of the NE experimental design, with animals immunised with formaldehyde NetB toxoid or NetB W262A on days 3, 9, and 15 post-hatching and infected with C. perfringens strain 56 on days 17, 18, 19, and 20; animals were culled, necropsied, and scored for lesions within the small intestines on days 21, 22, or 23; and

FIG. 2 shows the lesion scores for poultry experimentally infected with C. perfringens, with control groups of untreated chickens or chickens dosed with adjuvant only, chickens immunised with NetB W262A or the alpha-toxin CPA247-370 alone and chickens immunised with a combination of NetB W262A and CPA247-370.

### EXAMPLES

Materials and Methods

Bacterial Strains and Plasmids

Plasmid pBAD (Invitrogen, Paisley, UK) was used as expression vector and Eschericia coli TOP10 (Invitrogen, Paisley, UK) as expression host. E. coli strains were grown either in Terrific Broth (TB) or Luria-Bertani (LB) agar supplemented with ampicillin (100 μg/ml) at 37° C. and shaken at 300 rpm, where appropriate.

The formulation of TB and LB was:

TB:

12 g tryptone

24 g yeast extract

4 ml glycerol

Adjust volume to 1000 ml with 100 ml of a filter sterilized solution of 0.17M KH₂PO₄ and 0.72M K₂HPO₄

LB:

10 g Bacto-tryptone

5 g yeast extract

10 g NaCl

Adjust pH to 7.5 with NaOH.

Adjust volume to 1000 ml with dH₂O

Animals and Housing Conditions

Ross 308 broiler chickens were obtained as one-day-old chicks from Vervaeke-Belavi Hatchery (Tielt, Belgium, BE3031) and the parent flock had not been vaccinated with the commercial Netvax or any other C. perfringens vaccine. All animals were housed in the same room. The birds were reared in pens at a density of 26-28 animals per 1.5 m² on wood shavings. All pens were separated by solid walls to prevent contact between birds from different treatment groups. Before the trial, the rooms were decontaminated with Metatectyl HQ (Clim'oMedic®, Metatecta, Belgium) and a commercial anticoccidial disinfectant (OOCIDE, DuPont Animal Health Solutions, Wilmington, USA). The chickens received ad libitum drinking water and feed. A 23 h/1 h light/darkness program was applied. The animal experiments were carried out according to the recommendations and following approval of the Ethical Committee of the Faculty of Veterinary Medicine, Ghent University, Belgium.

Expression and Purification of NetB W262A

The expression and purification of NetB W262A with N-terminal His-tags (SEQ ID NO:19) for purification, was carried out in E. coli TOP10 cells and purification was carried out as described previously (Fernandes da Costa et al., Vaccine 2013 Aug. 20; 31(37):4003-8). In summary, the recombinant E. coli carrying the pBAD-NetB expression vector was grown in TB to an optical density (OD_{595 nm}) of 0.5 and expression of the toxin induced for 6 h by adding arabinose at a final concentration of 0.02% (w/v). Bacterial cells were harvested by centrifugation, lysed enzymatically using BugBuster (Invitrogen, Paisley, UK), and NetB purified by Ni-NTA chromatography columns (GE Healthcare Life Sciences, Little Chalfont, UK) according to the manufacturer's instructions. The protein was transferred into Tris-buffered saline (TBS; 20 mM Tris pH 7.5, 150 mM NaCl) using PD-10 desalting columns (GE Healthcare Life Sciences, Little Chalfont, UK) and protein concentrations measured with a UV-Vis spectrophotometer (Thermo Scientific, Cramlington, UK).

Expression and Purification of CPA247-370

Alpha-toxoid was produced and purified as described previously (Williamson et al., Vaccine 1993 September; 11(12):1253-8). In brief, recombinant E. coli containing the plasmid pGEX-3X-13, which encodes the C-terminal domain of the alpha-toxin fused to GST (GST-Cpa247-370; SEQ ID NO:20), was used. Bacteria were cultured in TB medium (100 μg/ml ampicillin) under gentle rotation at 37° C. Expression of alpha-toxoid was induced at an OD_{595 nm} of 0.5 for 6 h by the addition of IPTG (1 mM final concentration). Bacterial cells were harvested by centrifugation, lysed enzymatically using BugBuster (Invitrogen, Paisley, UK), and GST-Cpa₂₄₇₋₃₇₀ purified with prepacked GST GraviTrap columns (GE Healthcare Life Sciences, Little Chalfont, UK) according to the manufacturer's instructions. The eluted protein was dialysed against TB S by using PD-10 desalting columns (GE Healthcare Life Sciences, Little Chalfont, UK).

In Vivo NE Model

The NE model was based on the subclinical in vivo model described previously (Mot et al., Avian Pathol 2013; 42:2, 179-184). On days 3, 9, and 15, chickens were each immunised with 30 μg of either NetB W262A or CPA247-370 or both. Quil-A (50 Brenntag Biosector, Frederikssund, Denmark) was used as an adjuvant. The mixture was diluted in PBS to a total volume of 200 μl, mixed well by vortexing and filter-sterilised (0.2 μm pore size). Birds were vaccinated subcutaneously in the neck with a 200 μl dose. Controls consisted of an untreated group and a group receiving only the Quil-A (50 μg) adjuvant. Sera samples were taken on day 16 and on days 21, 22 and 23.

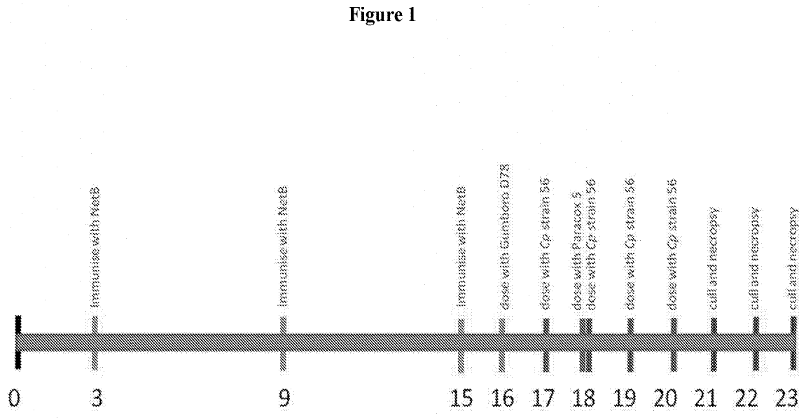

Nobilis Gumboro D 78 vaccine (Schering-Plough Animal Health, Brussels, Belgium) was given in the drinking water on day 16. From day 17 onwards, soy bean meal was replaced by fishmeal (30%) as a protein source. All groups were challenged orally, using a plastic tube inserted in the crop, on days 17, 18, 19 and 20 with a single dose of approximately 4×10⁸ cfu of C. perfringens strain 56 (Fernandes da Costa et al., Vaccine 2013 Aug. 20; 31(37):4003-8). On day 18, all animals were orally inoculated with a 10× dose of Paracox-5 (Schering-Plough Animal Health, Brussels, Belgium) (Mot et al., Avian Pathol 2013; 42:2, 179-184). On days 21, 22, and 23, one-third of the birds in each group were euthanised and necropsied. A schematic outline of the experimental design is shown in FIG. 1.

Measurement of Antibody to NetB and Alpha-Toxin Using ELISA

Sera samples were taken on day 16 and on day 21-22-23.

Assessment of Protection

NE severity was assessed by scoring lesions within the small intestine of each animal (duodenum to ileum) as described by Keyburn et al., Infect Immun 2006 November; 74(11):6496-500, as follows: 0=no gross lesions; 1=congested intestinal mucosa; 2=focal necrosis or ulceration (1-5 foci); 3=focal necrosis or ulceration (6-15 foci); 4=focal necrosis or ulceration (>16 foci); 5=patches of necrosis 2-3 cm long; 6=diffuse necrosis typical of field cases. Animals showing lesion scores of 2 or higher were classified as NE positive.

Statistical Analysis

For the in vivo NE model, differences within the occurrence of NE-positive animals between the controls and the NetB toxoid vaccinated groups were evaluated by a binary logistic regression analysis with the SPSS Statistics software 21.0 (SPSS Inc., Chicago, USA). A 2-way ANOVA analysis with the GraphPad Prism software 5.01 (GraphPad Software, La Jolla, USA) was used to compare the means of ELISA data. In both analysis, a p value of less than 0.01 was considered as significant (***: p<0.001; **: p<0.01).

Cytotoxicity Assay for Effect of rNetB on LMH Cells

Cytotoxicity is measured using the CytoTox96® kit (Promega), in which cytotoxicity is measured as the amount of LDH (lactate dehydrogenase) released from the cell cytosol into the medium caused by the presence of the tested pore-forming cytotoxic compound. A polypeptide is evaluated for its cytotoxicity by incubation with chicken hepatocellular carcinoma (LMH; ATTC: CRL-2117) cells, a cell line known to be susceptible for the toxin.

LMH cells are grown in Waymouth's MB 752/1 medium (Invitrogen) supplemented with 10% fetal calf serum at 37° C. in a 5% CO₂ incubator to 70-80% confluency in 96-well plates. Cells are incubated with serial dilutions of NetB in Waymouth's medium (100 μl final volume in each well) for 2 h at 37° C. Control cells are incubated with Waymouth's medium to determine either the base line (0%) or total cell lysis (100%), achieved by freezing and thawing of the cells. After 2 h of incubation the supernatant is assayed and percentage cytotoxicity was determined relative to the control groups.

Results

Protection Against Experimental NE after Immunisation with NetB W262A and CPA247-370

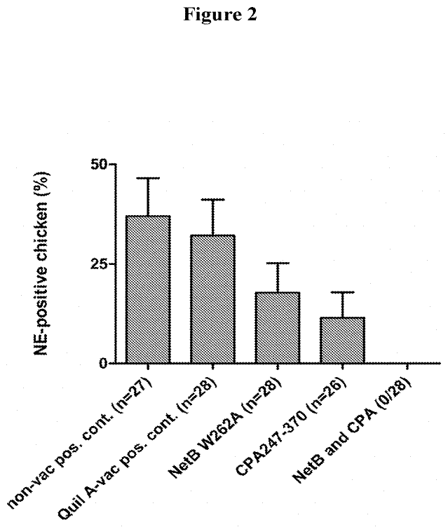

Immunisation with NetB W262A and CPA247-370 reduced lesion scores completely to the control groups in poultry experimentally infected with C. perfringens. In the control groups of untreated chickens, or chickens dosed with adjuvant only, the percentage positive chickens were 37% and 32%, respectively. In contrast, in the group immunised with NetB W262A 18% of the chickens showed lesions and in animals immunised with CPA247-370 12% of the chickens were NE-positive. None of the animals vaccinated with a combination of NetB W262A and CPA247-370 developed NE lesions (FIG. 2).

1. A method of (i) eliciting an immune response against C. perfringens, and/or (ii) vaccinating a subject against infection by C. perfringens, comprising administering to the subject a protective amount of a composition comprising:
a) a reduced toxicity necrotic enteritis toxin B (NetB) epitope polypeptide comprising amino acid sequence SEQ ID NO: 2, or comprising a functional fragment of SEQ ID NO: 2 having at least about 95% global sequence identity to SEQ ID NO: 2 and comprising ETTQXRGTNK (SEQ ID NO:10), wherein X is any amino acid other than W; and
b) a reduced toxicity C. perfringens alpha-toxin epitope polypeptide comprising the amino acid sequence SEQ ID NO: 3, or comprising a functional fragment of SEQ ID NO: 3 having at least about 95% global sequence identity to SEQ ID NO: 3,
wherein the composition provides protection against necrotic enteritis caused by C. perfringens.

2. The method according to claim 1, wherein said composition comprises a polynucleotide encoding said reduced toxicity NetB epitope polypeptide and reduced toxicity C. perfringens alpha-toxin epitope polypeptide and/or a vector comprising said polynucleotide and/or a cell comprising said composition, optionally wherein said cell is a Salmonella, Bacillus, Lactococcus or Lactobacillus cell.

3. The method according to claim 1, wherein said composition comprises at least one excipient and/or diluent.

4. The method according to claim 1, wherein the reduced toxicity NetB epitope polypeptide comprises at least one of the sequences:
a) YHAIXGNQLF (SEQ ID NO: 4), wherein X is any amino acid other than Y;
b) FMKSXLYNNG (SEQ ID NO: 6), wherein X is any amino acid other than R; or
c) YILNXETTQW (SEQ ID NO: 8), wherein X is any amino acid other than W.

5. The method according to claim 4, wherein any X is A.

6. The method according claim 1, wherein the alpha-toxin epitope polypeptide comprises a sequence of at least 5 contiguous amino acids from SEQ ID NO: 18.

7. The method according to claim 1, wherein the alpha-toxin epitope polypeptide comprises the amino acid sequence SEQ ID NO: 12 and/or 14.

8. The method according to claim 1, wherein the alpha-toxin epitope polypeptide comprises the amino acid sequence SEQ ID NO:16.

9. The method according to claim 1, wherein the alpha-toxin epitope polypeptide comprises the amino acid sequence SEQ ID NO:18, or a functional fragment or variant thereof having at least 34% global sequence identity to SEQ ID NO:18.

10. The method according to claim 1, wherein at least one of a) or b) is a recombinant polypeptide.

11. The method according to claim 10, wherein the recombinant protein is a fusion protein comprising GST or a His-tag.

12. The method according to claim 10, wherein the recombinant protein comprises a) fused to b) or comprises a fusion protein comprising a) and b).

13. The method according to claim 1, wherein said composition comprises a subunit vaccine.

14. The method according to claim 1, wherein said composition is animal feed or is administered in animal feed.

15. The method according to claim 1, wherein the subject is poultry.

16. The method according to claim 15, wherein the subject is of the genus Gallus.

17. The method according to claim 15, wherein the subject is of the species Gallus gallus.

18. The method according to claim 1, wherein a) and b) are administered sequentially to the subject.


SEQUENCE LISTING (21 sequences)

SEQ ID NO: 1 - protein, 292 aa, Clostridium perfringens
SELNDINKIELKNLSGEIIKENGKEAIKYTSSDTASHKGWKATLSGTFIEDPHSDKKTALLNLEGFIPSDKQIFGSKYYGKMKWPETYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQWRGTNKLSSTSEYNEFMFKINWQDHKIEYYL

SEQ ID NO: 2 - protein, 292 aa, Artificial Sequence
  Substituted sequence
SELNDINKIELKNLSGEIIKENGKEAIKYTSSDTASHKGWKATLSGTFIEDPHSDKKTALLNLEGFIPSDKQIFGSKYYGKMKWPETYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQARGTNKLSSTSEYNEFMFKINWQDHKIEYYL

SEQ ID NO: 3 - protein, 124 aa, Clostridium perfringens
NDPSVGKNVKELVAYISTSGEKDAGTDDYMYFGIKTKDGKTQEWEMDNPGNDFMTGSKDTYTFKLKDENLKIDDIQNMWIRKRKYTAFSDAYKPENIKIIANGKVVVDKDINEWISGNSTYNIK

SEQ ID NO: 4 - protein, 10 aa, Artificial Sequence
  Mutant peptide fragment
  Xaa (5)..(5): Any amino acid other than Y
YHAIXGNQLF

SEQ ID NO: 5 - protein, 20 aa, Artificial Sequence
  Mutant peptide fragment
  Xaa (10)..(10): Any amino acid other than Y
YNIDSYHAIXGNQLFMKSRL

SEQ ID NO: 6 - protein, 10 aa, Artificial Sequence
  Mutant peptide fragment
  Xaa (5)..(5): Any amino acid other than R
FMKSXLYNNG

SEQ ID NO: 7 - protein, 20 aa, Artificial Sequence
  Mutant peptide fragment
  Xaa (10)..(10): Any amino acid other than R
YGNQLFMKSXLYNNGDKNFT

SEQ ID NO: 8 - protein, 10 aa, Artificial Sequence
  Mutant peptide fragment
  Xaa (5)..(5): Any amino acid other than W
YILNXETTQW

SEQ ID NO: 9 - protein, 20 aa, Artificial Sequence
  Mutant peptide fragment
  Xaa (10)..(10): Any amino acid other than W
RFDNDYILNXETTQWRGTNK

SEQ ID NO: 10 - protein, 10 aa, Artificial Sequence
  Mutant peptide fragment
  Xaa (5)..(5): Any amino acid other than W
ETTQXRGTNK

SEQ ID NO: 11 - protein, 20 aa, Artificial Sequence
  Mutant peptide fragment
  Xaa (10)..(10): Any amino acid other than W
YILNWETTQXRGTNKLSSTS

SEQ ID NO: 12 - protein, 5 aa, Artificial Sequence
  Protein fragment
TDDYM

SEQ ID NO: 13 - protein, 10 aa, Artificial Sequence
  Protein fragment
DAGTDDYMYF

SEQ ID NO: 14 - protein, 5 aa, Artificial Sequence
  Protein fragment
NPGND

SEQ ID NO: 15 - protein, 10 aa, Artificial Sequence
  Protein fragment
EMDNPGNDFM

SEQ ID NO: 16 - protein, 32 aa, Artificial Sequence
  Protein fragment
DAGTDDYMYFGIKTKDGKTQEWEMDNPGNDFM

SEQ ID NO: 17 - protein, 10 aa, Artificial Sequence
  Mutant peptide fragment
ETTQARGTNK

SEQ ID NO: 18 - protein, 370 aa, Clostridium perfringens
WDGKIDGTGTHAMIVTQGVSILENDLSKNEPESVRKNLEILKENMHELQLGSTYPDYDKNAYDLYQDHFWDPDTDNNFSKDNSWYLAYSIPDTGESQIRKFSALARYEWQRGNYKQATFYLGEAMHYFGDIDTPYHPANVTAVDSAGHVKFETFAEERKEQYKINTAGCKTNEAFYTDILKNKDFNAWSKEYARGFAKTGKSIYYSHASMSHSWDDWDYAAKVTLANSQKGTAGYIYRFLHDVSEGNDPSVGKNVKELVAYISTSGEKDAGTDDYMYFGIKTKDGKTQEWEMDNPGNDFMTGSKDTYTFKLKDENLKIDDIQNMWIRKRKYTAFSDAYKPENIKIIANGKVVVDKDINEWISGNSTYNIK

SEQ ID NO: 19 - protein, 331 aa, Artificial Sequence
  Expression construct
MGGSHHHHHHGMASMTGGQQMGRDLYDDDDKDRWGSELESELNDINKIELKNLSGEIIKENGKEAIKYTSSDTASHKGWKATLSGTFIEDPHSDKKTALLNLEGFIPSDKQIFGSKYYGKMKWPETYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQARGTNKLSSTSEYNEFMFKINWQDHKIEYYL

SEQ ID NO: 20 - protein, 353 aa, Artificial Sequence
  Expression construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLIEGRGIPGNDPSVGKNVKELVAYISTSGEKDAGTDDYMYFGIKTKDGKTQEWEMDNPGNDFMTGSKDTYTFKLKDENLKIDDIQNMWIRKRKYTAFSDAYKPENIKIIANGKVVVDKDINEWISGNSTYNIK

SEQ ID NO: 21 - protein, 6 aa, Artificial Sequence
  Linker
GSGGGG